(19) United States
(12) Patent Application Publication
(10) Pub. No.: US 2006/0184186 A1
(43) Pub. Date: Aug. 17, 2006

(54) Drilling guidewire for treating chronic total occlusion

(75) Inventor: Michael S. Noone, Londonderry, NH (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/059,206

(22) Filed: Feb. 16, 2005

(51) Int. Cl. A61B 17/22; A61M 25/00

(52) U.S. Cl. 606/159; 600/585

(57) ABSTRACT

A rotational drilling guidewire includes an elongate body portion and a drilling portion. The drilling portion has multiple cutting elements and is sized to bore an opening having a diameter substantially the same as that of the elongate body portion. A system for treating a vascular condition includes a rotational drilling guidewire and a rotator. The rotator is capable of rotating the guidewire at speeds greater than can be achieved by manual rotation of the guidewire. A method of treating an occluded vessel comprises delivering a guide catheter adjacent to an occlusion, passing a drilling guidewire through the guide catheter to a position abutting the occlusion, rotating the guidewire to bore an opening the same diameter as the guidewire through the occlusion.

### TECHNICAL FIELD

This invention relates generally to medical devices that are used for treating vascular conditions. More specifically, the invention relates to a drilling guidewire for use in treating chronic total occlusion.

### BACKGROUND OF THE INVENTION

Guidewires are conventionally used to guide medical instruments to a desired treatment location within a patient's vasculature. In a typical procedure, the clinician forms an access point for the guidewire by creating an opening in a peripheral blood vessel, such as the femoral artery. The flexible guidewire is then introduced through the opening and is advanced by the clinician through the patient's blood vessels until the guidewire extends across a lesion to be treated. A treatment catheter, such as a balloon catheter for a percutaneous transluminal coronary angioplasty (PTCA) or a catheter carrying a stent, may then be inserted over the guidewire and similarly advanced through vasculature until it reaches the treatment site.

Chronic total occlusions (CTOs), encountered in 10% to 20% of all interventional procedures, present significant problems for a clinician attempting to perform PTCA and other wire-guided procedures. These complete blockages in the arteries commonly have a calcified fibrous cap that obstructs passage of a conventional guidewire. Because a guidewire must be passed through the blockage before a treatment catheter can be placed across the lesion, failure to place the guidewire may result in a patient undergoing coronary bypass surgery rather than a less invasive procedure.

Therefore, it would be desirable to provide a device, system, and method suitable for treating chronic total occlusions that overcome the aforementioned and other disadvantages.

### SUMMARY OF THE INVENTION

One aspect of the present invention is a rotational drilling guidewire, comprising an elongate body portion and a drilling portion. The drilling portion has a plurality of cutting elements and is sized to bore an opening having a diameter substantially the same as that of the elongate body portion.

Another aspect of the present invention is a system for treating a vascular condition, comprising a rotational drilling guidewire and a guidewire rotator. The guidewire includes an elongate body portion and a drilling portion. The drilling portion has a plurality of cutting elements and is sized to bore an opening having a diameter substantially the same as that of the elongate body portion. A proximal portion of the rotational drilling guidewire is removably attached to the guidewire rotator.

Yet another aspect of the present invention is a method of treating an occluded vessel. A guide catheter is delivered to a position adjacent to an occlusion in a vessel. A rotational drilling guidewire having an elongate body portion and a drilling portion is passed through the guide catheter to a position in which the drilling portion abuts the occlusion. The guidewire is rotated to bore an opening through the occlusion, the opening having a diameter substantially the same as the diameter of the elongate body portion. The guidewire is then extended through the bored opening.

The aforementioned and other features and advantages of the invention will become further apparent from the following detailed description of the presently preferred embodiments, read in conjunction with the accompanying drawings, which are not to scale. The detailed description and drawings are merely illustrative of the invention rather than limiting, the scope of the invention being defined by the appended claims and equivalents thereof.

### BRIEF DESCRIPTION OF THE DRAWINGS

FIG. 1 is a side view of one embodiment of a rotational drilling guidewire, in accordance with the present invention;

FIG. 2 is an end view of the guidewire of FIG. 1;

FIG. 3 is a side view of one embodiment of a system for treating a vascular condition, in accordance with the present invention;

FIG. 4 is a flow diagram of one embodiment of a method of treating an occluded vessel, in accordance with the present invention.

The same reference numbers are used throughout the drawings to refer to the same parts.

### DETAILED DESCRIPTION OF THE PRESENTLY PREFERRED EMBODIMENTS

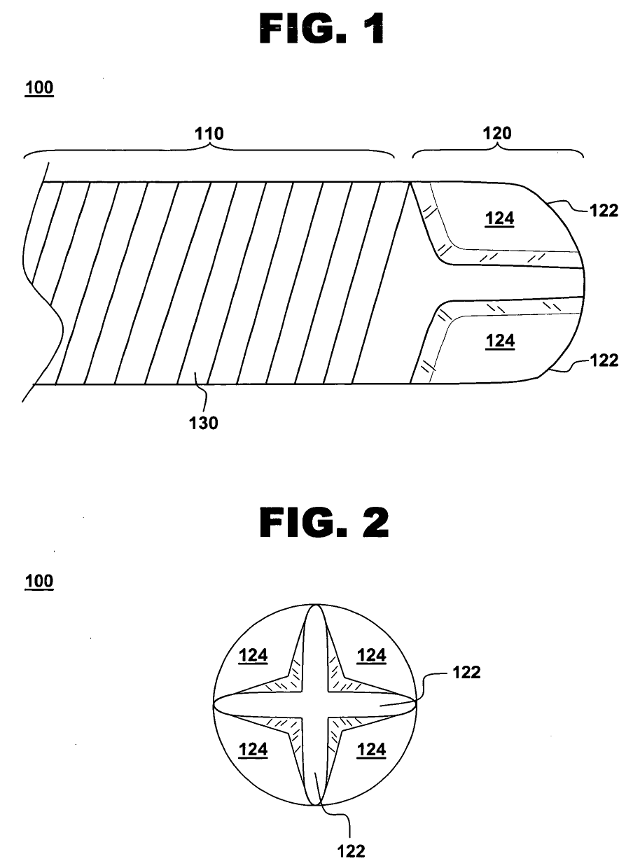

One aspect of the present invention is a rotational drilling guidewire. One embodiment of the device, in accordance with the present invention, is illustrated in FIG. 1 at 100. Guidewire 100 comprises elongate body portion 110 and drilling portion 120. Spring coil 130 surrounds a core within distal section of body portion 110.

Rotational drilling guidewire 100 is formed using a biocompatible material such as stainless steel, nitinol or age-hardenable nickel-cobalt-chromium-molybdenum alloy. Where the guidewire is to be used during a procedure such as a conventional percutaneous transluminal coronary angioplasty (PTCA) involving, femoral artery access, guidewire 100 may be about 120 centimeters to about 300 centimeters long, with a length of about 180 centimeters often being used. The outer diameter of the guidewire may range from about 0.010 inches to 0.038 inches, and preferably is about 0.014 inches.

Body portion 110 comprises the elongate proximal portion of the guidewire, while drilling portion 120 forms the distal tip of guidewire 100. As used herein, the terms “distal” and “proximal” are with reference to the treating clinician during deployment of the guidewire. “Distal” indicates a portion distant from, or a direction away from, the clinician; and “proximal” indicates a portion near to, or a direction toward, the clinician.

A distal section of body portion 110 may be ground down or otherwise reduced to a smaller diameter than the rest of the body portion. The reduced diameter core section provides increased flexibility for maneuvering the guidewire through a vessel to a treatment site. Spring coil 130 encases this core section, maintaining the flexibility of the core section while returning the section to approximately the same diameter as the remainder of the guidewire. The spring coil both prevents the core from cutting or otherwise damaging the walls of the vessel and reinforces the core, ensuring adequate pushability for the guidewire.

Drilling portion 120 is sized to bore an opening having a diameter substantially the same as the diameter of body portion 110. In the present embodiment, drilling portion 120 is approximately 1 to 10 millimeters in length and has a diameter approximately equal to that of body portion 110.

Drilling portion 120 has at least two cutting elements. In the present embodiment, drilling portion 120 is symmetrical; i.e., the portion displays bilateral symmetry from at least two perspectives, as seen in FIG. 1 and also in FIG. 2, which shows a distal end view of guidewire 100. Drilling portion 120 is fluted, having two longitudinal flutes 122 framed by four areas of relief 124, one on either side of each of the two flutes. The flutes are thin, blade-like structures that, when the guidewire is rotated, act as cutting elements to allow drilling portion 120 to bore through an obstruction such as a chronic total occlusion. The guidewire may be rotated manually or using a mechanical rotator such as is described below. The areas of relief may be formed using, for example, a hydraulic press with a fixture shaped to press indentations into the distal end of guidewire 100. One skilled in the art will appreciate that other methods, including metal cutting, electric discharge machining (EDM) and metal injection molding (MIM), may be used to form two or more longitudinal flutes or other symmetrical structures into the distal tip of the guidewire.

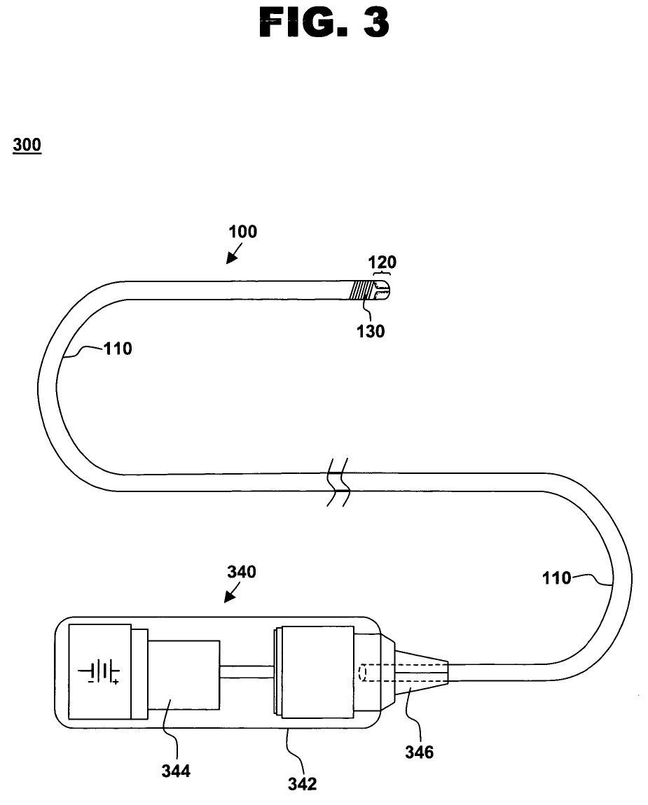

Another aspect of the present invention is a system for treating a vascular condition. One embodiment of the system, in accordance with the present invention, is illustrated in FIG. 3 at 300. System 300 comprises rotational drilling guidewire 100, described above, and a guidewire rotator 340. In another embodiment, the system may include an alternative rotational drilling guidewire that is in accordance with the present invention.

As described more fully above, drilling guidewire 100 comprises a stainless steel, nitinol or age-hardenable nickel-cobalt-chromium-molybdenum alloy wire and includes elongate body portion 110 and drilling portion 120. The drilling portion is symmetrical and has a plurality of cutting elements comprising flutes framed by areas of relief. The drilling portion is sized to bore an opening having a diameter substantially the same as that of the elongate body portion. A distal section of body portion 110 has a reduced diameter core surrounded by spring coil 130.

As illustrated in FIG. 3, a proximal portion of rotational drilling guidewire 100 is attached to guidewire rotator 340. The guidewire is removable from the guidewire rotator. Guidewire rotator 340 is similar to a conventional electric drill, with guidewire 100 serving a function similar to that of a drill bit. In the present embodiment, guidewire rotator 340 includes housing 342 to receive the guidewire and motor 344 to rotate the guidewire. Guidewire rotator 340 is shown partially cut away to reveal motor 344 positioned within housing 342.

Rotator 340 remains outside the patient, allowing housing 342 to be made using any of a wide variety of materials, including metals and plastics. One skilled in the art will appreciate that housing 342 may also assume a wide variety of shapes, the shape not being limited to that shown in FIG. 3. Motor 344 is preferably an electric motor powered by, for example, a battery, as shown, or by an alternating current source. In the present embodiment, rotator 340 is capable of rotating guidewire 100 at high speeds, meaning speeds that are, at a minimum, greater than can be achieved by manual rotation of the guidewire.

Guidewire 100 is removable from rotator 340 and may be advanced through a patient's vasculature prior to attaching the guidewire to the rotator. Once guidewire 100 has been delivered to a position adjacent to an obstruction, for example a chronic total occlusion, a proximal portion of guidewire 100 may be inserted into rotator 340 and retained within the rotator using, for example, a clamp, a chuck, or a collet, as is indicated in FIG. 3 at 346. The rotator motor may then be activated to cause entire guidewire 100 to rotate. The rotation of drilling portion 120 causes the portion to bore through the obstruction, allowing guidewire 100 to be extended beyond the obstruction.

In the present embodiment, guidewire 100 is preferably rotated in a direction to cause spring coil 130 to tighten against itself rather than unwinding. In an alternative embodiment that does not include a spring coil, a drilling portion such as that shown in FIGS. 1 and 2 may be rotated clockwise and/or counterclockwise, the drilling efficiency of portion 120 being essentially the same in either direction. In other embodiments, the drilling portion may be designed to bore more efficiently in one direction than in another, as is the case with most conventional drill bits.

System 300 may also include a supporting catheter (not shown) to provide additional stiffness for guidewire 100 while the guidewire is being pushed and rotated through an obstruction. The supporting catheter is made from a relatively stiff material such as polyimide and has an inner diameter sized to receive guidewire 100. The supporting catheter is placed over guidewire 100 with drilling portion 120 extending out of the distal end of the catheter. Once an opening has been bored through the obstruction, the supporting catheter may be withdrawn and guidewire 110 extended beyond the obstruction. Alternatively, the supporting catheter may remain in place within the guide catheter while guidewire 110 is extended out from the distal end of the supporting catheter. It will be apparent to one skilled in the art that a supporting catheter is not a required element of the system. The guidewire may be designed to have sufficient stiffness to bore through an obstruction without assistance from a supporting catheter.

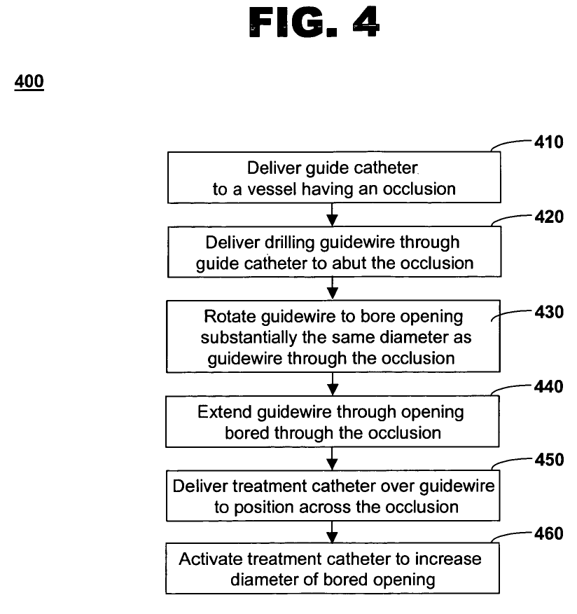

Yet another aspect of the present invention is a method of treating an occluded vessel. FIG. 4 shows a flow diagram of one embodiment of the method in accordance with the present invention.

A guide catheter is delivered to a vessel having an occlusion (Block 410). In the present embodiment, the guide catheter is advanced to the ostium of a coronary artery.

A rotational drilling guidewire having an elongate body portion and a drilling portion is delivered through the guide catheter to a position in which the drilling portion abuts the occlusion (Block 420). In the present embodiment, the guidewire illustrated in FIGS. 1 and 2 at 100 and described above is advanced through the guide catheter and extended beyond the distal end of the guide catheter until it abuts a chronic total occlusion (CTO) in the coronary artery.

The guidewire is rotated to bore an opening through the occlusion, the opening having a diameter substantially the same as the diameter of the elongate body portion (Block 430). The guidewire may be rotated manually by the clinician. Preferably, the guidewire is rotated using a device having a motor capable of rotating the guidewire at a high speed, meaning a speed that is, at a minimum, faster than can be achieved by manual rotation of the guidewire. One such device is described above and illustrated in FIG. 3 at 340.

The guidewire is then extended through the bored opening (Block 440). The guidewire extends far enough beyond the occlusion to provide direction and stability for a treatment catheter that is delivered over the guidewire to a position across the occlusion (Block 450).

Once the treatment catheter has been placed across the occlusion, it may be activated to increase the diameter of the opening that has been bored through the occlusion (Block 460). For example, the treatment catheter may carry a percutaneous transluminal coronary angioplasty (PTCA) balloon, with the size of the opening being increased when the balloon is inflated.

While the embodiments of the invention disclosed herein are presently considered to be preferred, various changes and modifications can be made without departing from the spirit and scope of the invention. The scope of the invention is indicated in the appended claims, and all changes and modifications that come within the meaning and range of equivalents are intended to be embraced therein.

What is claimed is:

1. A rotational drilling guidewire, comprising:
a flexible elongate body portion; and
a drilling portion having a plurality of cutting elements, the drilling portion being sized to bore an opening having a diameter substantially the same as the diameter of the elongate body portion.

2. The guidewire of claim 1, wherein the drilling portion includes a plurality of flutes and a plurality of areas of relief.

3. The guidewire of claim 1, wherein the drilling portion is symmetrical.

4. The guidewire of claim 1, wherein a distal section of the elongate body portion has a reduced diameter core.

5. The guidewire of claim 4 further comprising:
a spring coil surrounding the core.

6. The guidewire of claim 1 wherein the guidewire comprises one of a stainless steel, a nitinol, or an age-hardenable nickel-cobalt-chromium-molybdenum alloy wire.

7. A system for treating a vascular condition, comprising:
a rotational drilling guidewire including a flexible elongate body portion and a drilling portion having a plurality of cutting elements, the drilling portion being sized to bore an opening having a diameter substantially the same as the diameter of the elongate body portion; and
a guidewire rotator, wherein a proximal portion of the rotational drilling guidewire is removably attached to the guidewire rotator.

8. The system of claim 7, wherein the guidewire drilling portion includes a plurality of flutes and a plurality of areas of relief.

9. The system of claim 7, wherein the guidewire drilling portion is symmetrical.

10. The system of claim 7, wherein a distal section of the guidewire elongate body portion has a reduced diameter core.

11. The system of claim 10 wherein the guidewire further comprises a spring coil surrounding the core.

12. The system of claim 7 wherein the rotator includes a housing to receive a proximal portion of the rotational drilling guidewire and a motor to rotate the guidewire.

13. The system of claim 7 wherein the proximal portion of the rotational drilling guidewire is removably attached to the guidewire rotator using a fixture selected from a group consisting of a clamp, a chuck and a collet.

14. The system of claim 7 wherein the rotator is capable of rotating the guidewire at a high speed.

15. The system of claim 14 wherein the guidewire comprises one of a stainless steel, a nitinol, or an age-hardenable nickel-cobalt-chromium-molybdenum alloy wire.

16. A method of treating an occluded vessel, comprising:
delivering a guide catheter to a vessel having an occlusion;
passing a rotational drilling guidewire having a flexible elongate body portion and a drilling portion through the guide catheter to a position in which the drilling portion abuts the occlusion;
rotating the guidewire to bore an opening through the occlusion, the opening having a diameter substantially the same as the diameter of the elongate body portion; and
extending the guidewire through the opening.

17. The method of claim 16 further comprising:
delivering a treatment catheter over the guidewire to a position across the occlusion.

18. The method of claim 16 further comprising:
activating the treatment catheter to increase the diameter of the opening bored through the occlusion.

19. The method of claim 16 wherein the guidewire is rotated using a rotator.

20. The method of claim 19 wherein the rotator is capable of rotating the guidewire at a high speed.
